(19) United States
(12) Patent Application Publication
(10) Pub. No.: US 2004/0146854 A1
(43) Pub. Date: Jul. 29, 2004

(54) Attenuated forms of bovine viral diarrhea virus

(75) Inventors: Xuemei Cao, East Lyme, CT (US); Gabriele M. Zybarth, West Kingston, RI (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 09/989,933

(22) Filed: Nov. 21, 2001

Related U.S. Application Data
(60) Provisional application No. 60/252,513, filed on Nov. 22, 2000.
(60) Provisional application No. 60/256,515, filed on Dec. 18, 2000.

(51) Int. Cl. C12Q 1/70; C07H 21/04; C12N 7/01; C12N 15/86

(52) U.S. Cl. 435/5; 435/69.3; 435/235.1; 435/456; 435/325; 536/23.72

(57) ABSTRACT

The present invention relates to attenuated forms of bovine viral diarrhea (BVD) viruses. In particular, the present invention relates to attenuated BVD viruses made by mutating the N^{pro} protease gene and inserting a bovine ubiquitin coding sequence. The attenuated viruses of the present invention can be used to raise antibodies against BVDV. Immunogenic compositions and vaccine compositions, as well as therapeutic and diagnosis methods, are also provided by the present invention.

### FIELD OF THE INVENTION

The present invention relates to attenuated bovine viral diarrhea (BVD) viruses and methods of making the same by modifying the viral genome. The attenuated viruses, as well as the modified viral genome, can be used to produce antibodies against BVD virus or in vaccines designed to protect cattle from viral infection.

### BACKGROUND OF THE INVENTION

Bovine viral diarrhea (BVD) virus is classified in the pestivirus genus and Flaviviridae family. It is closely related to viruses causing border disease in sheep and classical swine fever. Infected cattle exhibit “mucosal disease” which is characterized by elevated temperature, diarrhea, coughing and ulcerations of the alimentary mucosa (Olafson, et al., Cornell Vet 36:205-213 (1946); Ramsey, et al., North Am. Vet 34:629-633 (1953)). The BVD virus is capable of crossing the placenta of pregnant cattle and may result in the birth of persistently infected (PI) calves (Malmquist, J. Am. Vet Med. Assoc. 152:763-768 (1968); Ross, et al., J. Am. Vet Med. Assoc. 188:618-619 (1986)). These calves are immunotolerant to the virus and persistently viremic for the rest of their lives. They provide a source for outbreaks of mucosal disease (Liess, et al., Dtsch. Tieraerztl. Wschr. 81:481-487 (1974)) and are highly predisposed to infection with microorganisms causing diseases such as pneumonia or enteric disease (Barber, et al., Vet Rec. 117:459-464 (1985)).

BVD viruses are classified as having one of two different biotypes. Those of the “cp” biotype induce a cytopathic effect in cultured cells, whereas viruses of the “ncp” biotype do not (Gillespie, et al., Cornell Vet. 50:73-79 (1960)). In addition, two major genotypes (type I and II) are recognized, both of which have been shown to cause a variety of clinical syndromes (Pellerin, et al., Virology 203:260-268 (1994); Ridpath, et al., Virology 205:66-74(1994)).

The genome of the BVD virus is approximately 12.5 kb in length and contains a single open reading frame located between the 5′ and 3′ non-translated regions (NTRs) (Collett, et al., Virology 165:191-199 (1988)). A polyprotein of approximately 438 kD is translated from this open reading frame and is processed into viral structural and nonstructural proteins by cellular and viral proteases (Tautz, et al., J. Virol. 71:5415-5422 (1997); Xu, et al., J. Virol. 71:5312-5322 (1997); Elbers, et al., J. Virol. 70:4131-4135 (1996); and Wiskerchen, et al., Virology 184:341-350 (1991)). Among the viral enzymes that participate in this processing are the proteases N^{pro} and NS3. N^{pro} is the first protein encoded by the viral open reading frame and cleaves itself from the rest of the synthesized polyprotein (Stark, et al., J. Virol. 67:7088-7093 (1993); Wiskerchen, et al., Virol. 65:4508-4514 (1991)).

Among the BVD vaccines that are currently available are those in which virus has been chemically inactivated (McClurkin, et al., Arch. Virol. 58:119 (1978); Fernelius, et al., Am. J. Vet Res. 33:1421-1431 (1972); and Kolar, et al., Am. J. Vet. Res. 33:1415-1420 (1972)). These vaccines have typically required the administration of multiple doses to achieve primary immunization, provide immunity of short duration and do not protect against fetal transmission (Bolin, Vet. Clin. North Am. Food Anim. Pract. 11:615-625 (1995)). In sheep, a subunit vaccine based upon a purified E2 protein has been reported (Bruschke, et al., Vaccine 15:1940-1945 (1997)). Unfortunately, only one such vaccine appears to protect fetuses from infection and this protection is limited to one strain of homologous virus. There is no correlation between antibody titers and protection from viral infection.

In addition, modified live virus (MLV) vaccines have been produced using BVD virus that has been attenuated by repeated passage in bovine or porcine cells (Coggins, et al., Cornell Vet 51:539 (1961); and Phillips, et al., Am. J. Vet Res. 36:135 (1975)) or by chemically induced mutations that confer a temperature-sensitive phenotype on the virus (Lobmann, et al., Am. J. Vet. Res. 45:2498 (1984); and Lobmann, et al., Am. J. Vet. Res. 47:557-561 (1986)). A single dose of MLV vaccine has proven sufficient for immunization and the duration of immunity can extend for years in vaccinated cattle (Coria, et al., Can. J. Con. Med. 42:239 (1978)). In addition, cross-protection has been reported from calves vaccinated with MLV-type vaccines (Martin, et al., In Proceedings of the Conference Res. Workers' Anim. Dis., 75:183 (1994)). However, safety considerations, such as possible fetal transmission of the virus, have been a major concern with respect to the use of these modified live viral vaccines (Bolin, Vet. Clin. NorthAm. Food Anim. Pract. 11:615-625 (1995)).

A clear need exists for new and effective vaccines to control the spread of the BVD virus. Given that the disease caused by this virus is one of the most widespread and economically important diseases of cattle, such vaccines would represent a substantial advance in livestock farming.

U.S. patent application Ser. No. 08/107,908 has described that the N^{pro} coding sequence or the N^{pro} protein of BVDV is not required for virus replication. The application has described the generation of an attenuated BVD virus, “BVDdN1”, in which the entire coding sequence for the N^{pro} protein has been deleted from the viral genome. BVDdN1 is infectious in tissue culture and elicits virus neutralizing serum antibodies when vaccinated into cows. Although BVDdN1 can be used as a vaccine against BVDV, BVDdN1 grows in tissue culture at a rate 2-log slower than the parent wild type virus, making the large-scale production of BVDdN1 difficult.

The present invention provides attenuated BVD virus carrying a deletion of only a portion of the N^{pro} coding sequence in the 3′ region of the N^{pro} gene, and an insertion of the coding region of a bovine ubiquitin gene. The attenuated BVD viruses of the present invention replicate faster than BVDdN1 which provides higher immunogenicity for protection and, which permits large-scale productions of more effective vaccines against BVDV infections.

### SUMMARY OF THE INVENTION

One embodiment of the present invention provides attenuated BVD viruses which carry in the viral genome, a mutated N^{pro} coding sequence having an intact 5′ region, and a sequence coding for a monomeric bovine ubiquitin, wherein the ubiquitin coding sequence is operably placed between the 3′ end of the mutated N^{pro} coding sequence and the 5′ end of the core protein coding sequence.

A preferred attenuated BVD virus of the present invention is BVDdN6, the genomic sequence of which is set forth in SEQ ID NO: 11. Attenuated viruses having a genomic sequence substantially the same as SEQ ID NO: 11 are also encompassed by the present invention.

Another embodiment of the present invention is directed to isolated genomic nucleic molecules of the attenuated BVD viruses as described above. Nucleic acid molecules as used herein encompass both RNA and DNA. A preferred nucleic acid molecule of the present invention is set forth in SEQ ID NO: 11. Nucleic acid molecules substantially the same as SEQ ID NO: 11 are also encompassed by the present invention.

In another embodiment, the present invention provides vectors carrying the genomic nucleic acid molecules of the present attenuated BVD viruses. A preferred vector is pBVDdN6 (ATCC No. PTA-2532) (SEQ ID NO: 12), in which the genomic sequence of BVDdN6 (SEQ ID NO: 11) has been inserted.

Still another embodiment of the present invention is directed to host cells into which the genomic nucleic acid molecule of an attenuated BVD virus of the present invention has been introduced. “Host cells” as used herein include both prokaryotic and eukaryotic cells.

Another embodiment of the present invention is directed to antibodies against BVDV made by infecting an animal with an effective dosage of any of the attenuated BVD viruses of the present, preferably, BVDdN6.

In another embodiment, the present invention provides a method of modifying a genome from an isolated wild type BVD virus to make it suitable for use in an immunogenic composition or a vaccine. According to this method, the genomic nucleic acid is modified to mutate the N^{pro} gene, and to insert a sequence coding for a monomeric bovine ubiquitin between the mutated N^{pro} coding sequence and the coding sequence of the core protein. The mutation of the N^{pro} gene renders the N^{pro} protein inactive, yet does not interfere with the function of the 5′ region of the N^{pro} gene, whose coding sequences are important to support viral protein translation initiation.

One embodiment of the present invention provides immunogenic compositions which include one or more of the attenuated BVD viruses of the present invention. A preferred attenuated BVD virus to be included in an immunogenic composition of the present invention is BVDdN6. Alternatively, the immunogenic compositions of the present invention can include genomic nucleic acid molecules of one or more of the attenuated BVD viruses of the present invention.

Another embodiment of the present invention provides methods of inducing an immune response against BVDV in an animal subject by administering an effective amount of an immunogenic composition of the present invention. “Animal subjects” as used herein include any animal that is susceptible to BVDV infections, such as sheep and swine.

In still another embodiment, the present invention provides vaccine compositions which include one or more of the attenuated BVD viruses of the present invention, preferably BDVdN6. Alternatively, the vaccine compositions can include the genomic nucleic acid molecules of one or more of the attenuated BVD viruses of the present invention.

In another embodiment, the present invention provides methods of treating BVDV infections in animal subjects by administering to an animal, a therapeutically effective amount of an attenuated BVD virus of the present invention. By “treating” is meant preventing or reducing the risk of infection by a virulent strain of BVDV (including both Type I and Type II), ameliorating the symptoms of a BVDV infection, or accelerating the recovery from a BVDV infection.

A further aspect of the present invention is directed to methods of determining the origin of a BVD virus in an animal subject, e.g., to determine the attenuated virus of a prior vaccination is the origin of a BVD virus in an animal. Such methods are based on the distinction of the attenuated BVD viruses of the present invention that are used in vaccines from wild type BVD strains in genomic composition and in protein expression. The methods of the present invention allow discrimination between vaccinated and infected animals, and permit the identification of the origin of a BVD virus in the event of alleged vaccine-associated outbreaks.

### BRIEF DESCRIPTION OF THE DRAWINGS

FIGS. 1A-1D graphically depicts the steps involved in the generation of plasmid pBVDdN6. First, the coding sequence of bovine ubiquitin gene was cloned into plasmid pvvNADLd1NS2 giving rise to plasmid pvvNADLd1ubiNS2 (FIG. 1A). From pvvNADLd1ubiNS2, a fragment containing the coding sequence for bovine ubiquitin and partial BVDV genomic sequences was moved into plasmid pNADLp15a (alternative infectious clone of BVDV) to obtain plasmid p15aD1ubiNS2 (FIG. 1B). Further modification of plasmid p15aD1ubiNS2 results in plasmid p15aDl (subviral replicon, FIG. 1C), which was subsequently used as the parent plasmid for the generation of plasmid pBVDdN6 (FIG. 1D).

FIG. 2A depicts the genomic sequence of BVDdN6 (SEQ. ID NO: 11) (nucleotide 1 represents the first nucleotide of the BVDV genome of its 5′ end).

FIG. 2B depicts the full sequence of a plasmid containing the complete BVDdN6 genomic sequence, designated at pBVDdN6 (SEQ. ID NO: 12).

FIG. 3 depicts the growth phenotype of the viruses BVDdN1, BVDdN6 and NADL (wild type) in MDBK cells in an immunohistochemistry assay.

FIG. 4 depicts the growth kinetics of the viruses BDVdN1, BDVdN6 and NADL (wild type) in MDBK cells.

### DETAILED DESCRIPTION OF THE INVENTION

It has been shown in the co-pending U.S. patent application Ser. No. 08/107,908, that the N^{pro} coding sequence or the N^{pro} protein of BVDV is not essential for replication of the virus. An attenuated BVDV virus (“BVDdN1”) has been described therein which carries a deletion of the full coding sequence for N^{pro} in the viral genome. BVDdN1 is less infectious than the parent wild type virus and elicits virus neutralizing serum antibodies when vaccinated into cows. The entire disclosure of U.S. patent application Ser. No. 08/107,908 is incorporated herein by reference. Although BVDdN1 can be used as a vaccine against BVDV, BVDdN1 grows in tissue culture at a rate about 2-log slower than the parent wild type virus, making the large-scale production of BVDdN1 difficult. Furthermore, the attenuated BVD virus of the present invention replicates faster than BVDdN1 which provides higher immunogenicity for protection.

The present inventors have discovered that less attenuated BVDV viruses can be produced by deleting only a portion of the N^{pro} coding sequence from the viral genome. Although not intending to be bound by any particular theory, the present inventors postulate that the dramatic reduction in the rate of viral replication of BVDdN1 as compared to the parent wild type virus is due to the deletion of genomic elements located within the 5′ region of the N^{pro} gene. These elements may contribute to the initiation of the translation process in the production of the viral polyprotein precursor. Thus, according to the present inventors, BVDV constructs which maintain at least a portion of the 5′ sequence of the N^{pro} coding region exhibit an increased efficiency in the translation of viral polyprotein precursors as compared to BVDdN1, and the viruses derived from such constructs replicate more efficiently than BVDdN1. However, N^{pro} is a protease required for the cleavage of the viral polyprotein precursor at a site between N^{pro} and the Core protein (C). A BVDV construct carrying a mutated N^{pro} coding sequence would then be translated into a polyprotein precursor having a mutated N^{pro} fused to the N-terminus of C, and such fusion would interfere with viral replication. According to the present invention, an intact N-terminus of C can be restored by inserting into the viral genome a coding sequence for bovine ubiquitin between a mutated N^{pro} coding sequence and the coding region of C. In a polyprotein precursor produced from such a chimeric viral genome, the N-terminal of bovine ubiquitin is linked to the C-terminal of the mutated N^{pro}, and the C-terminal Glycine 76 of bovine ubiquitin is fused to the first amino acid (Serine) at the N-terminal of C. Processing of the ubiquitin-C junction in a polyprotein precursor is mediated by cellular ubiquitin carboxyl-terminal hydrolases (UCH), which cleave ubiquitin directly after its C-terminal Glycine, giving rise to an intact N-terminus of C.

Accordingly, one embodiment of the present invention provides attenuated BVD viruses carrying in the viral genome, a mutated N^{pro} coding sequence having an intact 5′ region, and a sequence coding for a monomeric bovine ubiquitin, wherein the ubiquitin coding sequence is operably placed between the 3′ end of the mutated N^{pro} coding sequence and the 5′ end of the core protein coding sequence.

BVD “viruses”, “viral isolates” or “viral strains” as used herein refer to BVD viruses that consist of the viral genome, associated proteins, and other chemical constituents (such as lipids). Ordinarily, the BVD virus has a genome in the form of RNA. RNA can be reverse-transcribed into DNA for use in cloning. Thus, references made herein to nucleic acid and BVD viral sequences encompass both viral RNA sequences and DNA sequences derived from the viral RNA sequences. For convenience, genomic sequences of BVD as depicted in the SEQUENCE LISTING hereinbelow only refer to the DNA sequences. The corresponding RNA sequence for each is readily apparent to those of skill in the art.

An “attenuated virus” as used herein refers to a virus that replicates at a slower rate than its wild type counterpart. Whether a genetically engineered BVD virus is attenuated can be conveniently determined by comparing the growth of such virus with that of the parent wild type virus in cell lines susceptible to infection by the parent virus. Cell lines which can be employed for this purpose include, e.g., bovine testicular cell lines (RD), bovine kidney cell lines (MDBK), embryonic bovine trachea cells (EBTr) and bovine turbinate cells (BT-2).

By “intact 5′region” is meant a 5′ region which maintains the efficient translation initiation of viral proteins.

In accordance with the present invention, the N^{pro} coding sequence of the attenuated viruses carries a mutation in the 3′ region, and the 5′ region of the N^{pro} coding sequence remains intact. The term “5′region” and “3′region” as used herein refers to a region of the N^{pro} coding sequence that is proximate to the 5′ end and the 3′ end of the N^{pro} coding sequence, respectively. According to the present invention, the 5′ region of the N^{pro} coding sequence can encompass at least about 36 bases pairs, or preferably about 310 base pairs, from the 5′ end of the N^{pro} coding sequence.

The term “mutation” as used herein includes substitution, deletion or insertion of one or more base pairs which results in a substitution, deletion or insertion of one or more amino acid residues in the N^{pro} protein. According to the present invention, the mutation is sufficient to inactivate the function of the N^{pro} protein so as to keep the virus attenuated, and leaves the 5′ region of the N^{pro} gene intact so as to achieve a desirable rate of viral replication. Preferably, the mutation is a deletion of about 468 bp, more preferably about 194 bp, from the 3′ end of the N^{pro} coding sequence. A particularly preferred mutation is a deletion of one third of the N^{pro} coding region from the 3′ end.

The mutated N^{pro} coding sequence in the attenuated BVD viruses of the present invention is operably linked to a sequence coding for a monomeric bovine ubiquitin. By “operably linked” is meant that the ubiquitin coding sequence is linked to the mutated N^{pro} coding sequence in-frame such that in the resulting polyprotein precursor, the N-terminus of ubiquitin is fused to the C-terminus of the mutated N^{pro}.

The sequence coding for a monomeric bovine ubiquitin is, in turn, operably linked to the coding sequence for C in the viral genome. Similarly, by “operably linked” is meant that the ubiquitin coding sequence is linked to the C coding sequence in-frame such that in the resulting polyprotein precursor, the C-terminus of ubiquitin is fused to the N-terminus of C in the polyprotein precursor.

A preferred attenuated BVD virus of the present invention is BVDdN6. BVDdN6 carries in the genome a deletion (196 bp) of about one third of the coding region of the N^{pro} coding region (total 504 bp) from the 3′ end and an insertion of the coding region for the bovine ubiquitin downstream of the partial N^{pro} coding sequence and upstream of the coding sequence for the viral core protein (C). The genomic sequence of the BVDdN6 is set forth in SEQ ID NO: 11.

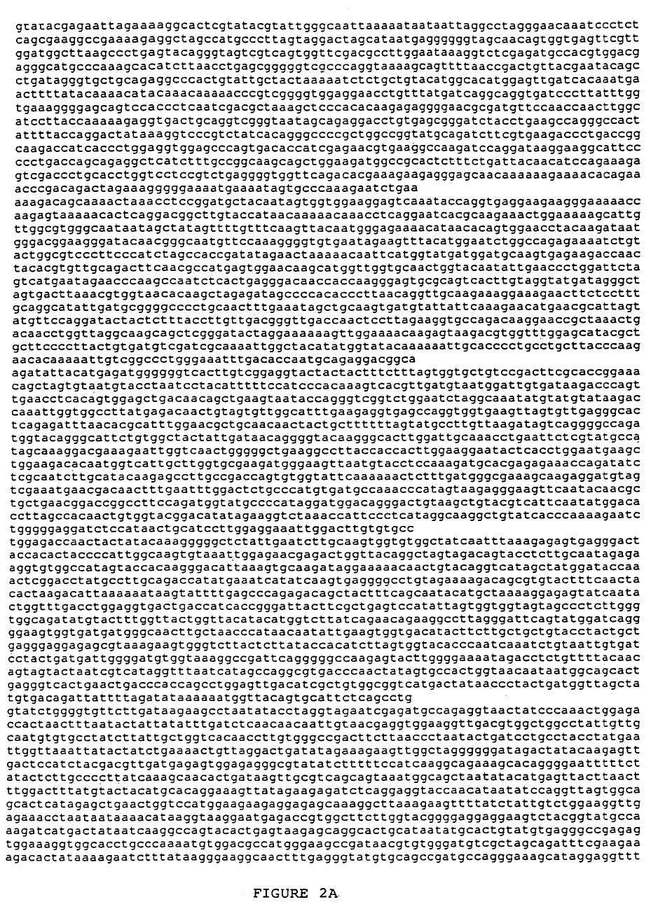

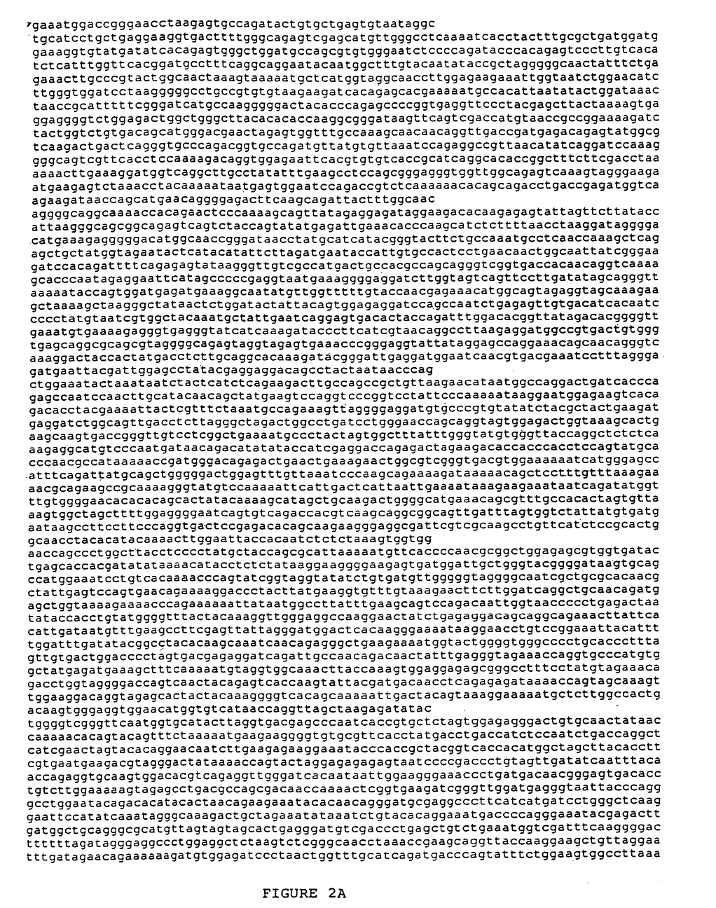

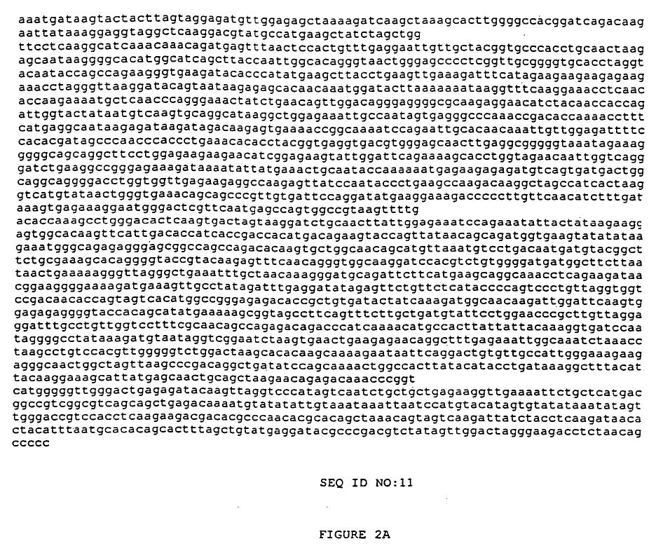

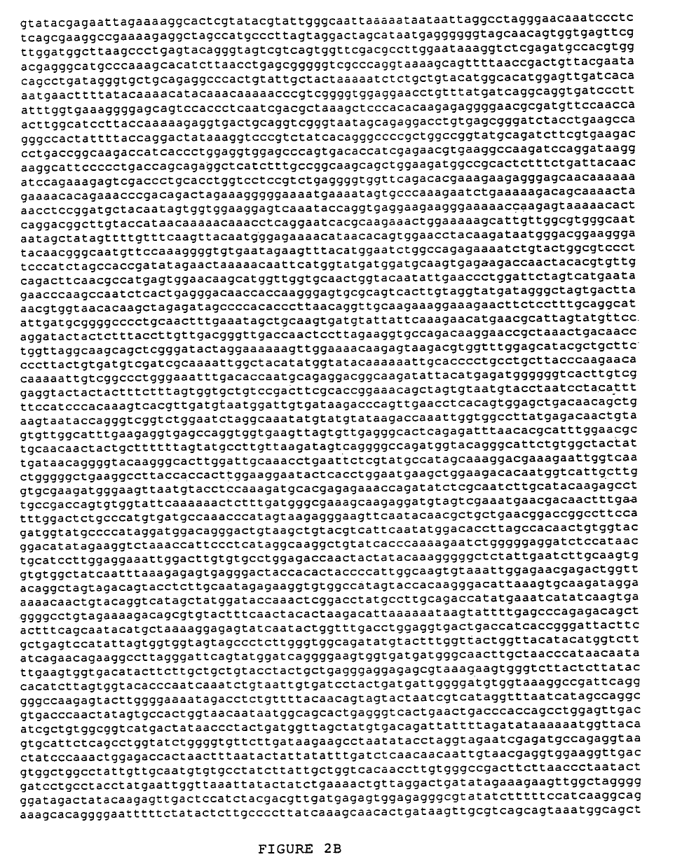

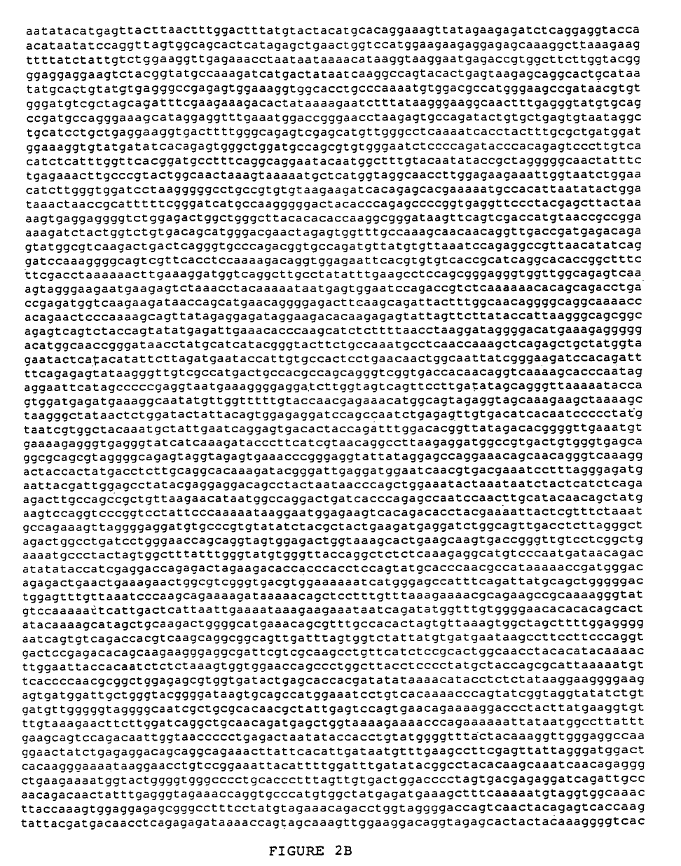

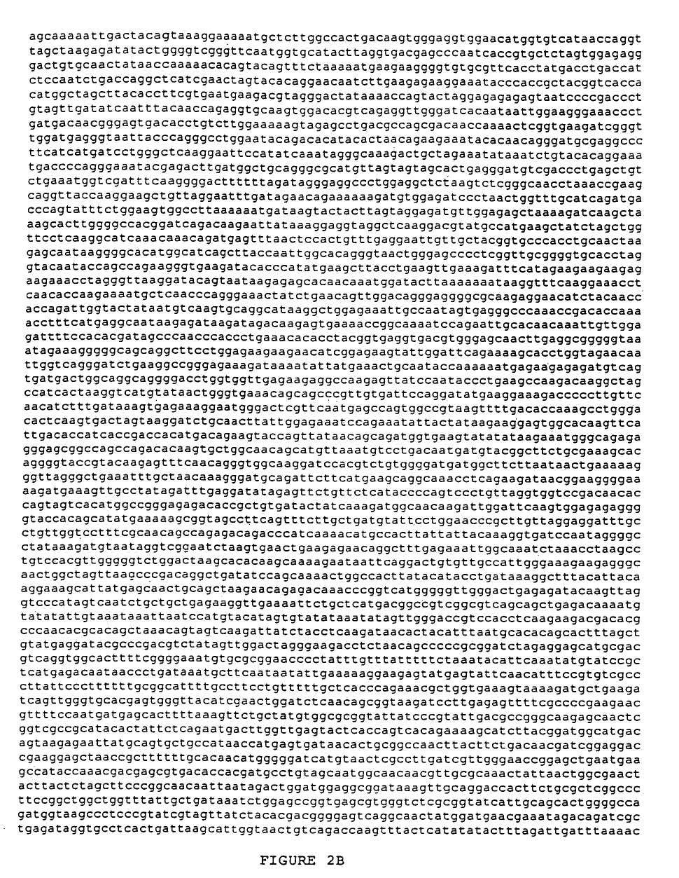

BVDdN6 has been generated as described in the Examples section below. Although this procedure can be used to obtain the virus, a plasmid containing the complete BVDdN6 genomic sequence, designated as pBVDdN6, has been deposited as ATCC No. PTA-2532 and represents the preferred source for isolating BVDdN6. The full sequence of pBVDdN6 is set forth in FIG. 2B and SEQ ID NO: 12. Standard procedures can be used to propagate and purify the plasmid. The preferred prokaryotic host cell for plasmid propagation is GM 2163 (available from NEB, U.S.A.), but other cell types can also be used. The plasmid can be introduced by transfection into eukaryotic host cells capable of supporting virus production, such as RD or MDBK cells. The virus can be produced in such host cells and isolated therefrom in highly purified form using known separation techniques such as sucrose gradient centrifugation, or ultra centrifugation precipitation.

The present invention also encompasses attenuated viruses having a genomic sequence substantially the same as SEQ ID NO: 11. Sequences that are substantially the same as SEQ ID NO: 11 may include, for example, degenerate nucleic acid sequences that encode the same BVD proteins as SEQ ID NO: 11, or sequences made by introducing into SEQ ID NO: 11, one or more insubstantial additions or substitutions. In particular, sequences carrying mutations that do not substantially alter the characteristics of BVDdN6 with respect to infectivity fall within the scope of the invention. The methods for introducing mutations into a given sequence are well known in the art.

Another embodiment of the present invention is directed to isolated genomic nucleic molecules of the attenuated BVD viruses as described above. Nucleic acid molecules as used herein encompass both RNA and DNA.

In this embodiment, the isolated genomic nucleic molecules of attenuated BVD viruses contain a mutated N^{pro} coding sequence having an intact 5′ region, and a sequence coding for a monomeric bovine ubiquitin, wherein the ubiquitin coding sequence is operably placed between the 3′ end of the mutated N^{pro} coding sequence and the 5′ end of the core protein coding sequence.

A preferred nucleic acid molecule of the present invention is SEQ ID NO: 11, setting forth the genomic sequence of BVDdN6. Nucleic acid molecules substantially the same as SEQ ID NO: 11 are also encompassed by the present invention.

In another embodiment, the genomic nucleic acid molecules of the present attenuated BVD viruses have been incorporated into appropriate vectors. The vectors carrying the genomic nucleic acid molecule of an attenuated BVD virus of the present invention can be introduced into appropriate host cells, either for the production of large amounts of the genomic nucleic acid molecules or for the production of progeny attenuated BVD viruses. The vectors may contain other sequence elements to facilitate vector propagation, isolation and subcloning; for example, selectable marker genes and origins of replication that allow for propagation and selection in bacteria and host cells. Preferred vectors for incorporation of BVD genomic sequences include PACY177 (New England, Biolabs, U.S.A.). A particularly preferred vector of the present invention is pBVDdN6 (ATCC No. PTA-2532), in which the genomic sequence of BVDdN6 (SEQ ID NO: 11) has been inserted (see FIG. 2B, Nv. 1-12617).

Still another embodiment of the present invention is directed to host cells into which the genomic nucleic acid molecule of an attenuated BVD virus of the present invention has been introduced. “Host cells” as used herein include any prokaryotic cells transformed with the genomic nucleic acid molecule, preferably provided by an appropriate vector, of an attenuated BVD virus. “Host cells” as used herein also include any eukaryotic cells infected with an attenuated BVD virus or otherwise carrying the genomic nucleic acid molecule of an attenuated BDV virus. For prokaryotic cells, the GM2rb3 strain of E. coli (NEB) has been found to give the best results for propagating the plasmid, and is generally preferred. For eukaryotic cells, mammalian cells such as MDBK cells (ATCC CCL 22) and RD cells (stable transformed bovine testicular cells) are generally preferred. However, other cultured cells can be used as well. The invention further includes progeny virus produced in such host cells.

Another embodiment of the present invention is directed to antibodies against BDV made by infecting an animal with an effective dosage of any of the attenuated BVD viruses of the present, preferably, BVDdN6. “An effective dosage” refers to a dosage high enough to provoke antibody production. “Antibodies against BVD virus” as used herein refer to antibodies that specifically recognize BVD viruses, preferably with at least about a 100-fold greater affinity for a strain of BVD virus than for any other, non-BVD virus.

Animals appropriate for use in making antibodies against BVD include any of the animals normally used for raising antibodies, such as mice, rabbits, goats, or sheep. Preferably, antibodies are made in cattle. Although not preferred, virus can be inactivated prior to administration to an animal using chemical treatments involving agents such as formalin, paraformaldehyde, phenol, lactopropionate, psoralens, platinum complexes, ozone or other viricidal agents. Compositions containing the virus can be administered to the animals by any route, but typically animals will be injected intramuscularly, subcutaneously or intravenously. Generally, the virus preparation will include an adjuvant, e.g. Freund's complete or incomplete adjuvant. Appropriate preparations for injection, injection schedules and the like are well known in the art and can be employed (see, e.g., Harlow et al., Antibodies, A Laboratory Manual, Cold Spring Harbor Laboratory, N.Y. (1988); Klein, Immunology: The Science of Self-Nonself Discrimination (1982)). Monoclonal antibodies can also be prepared using standard procedures (Kennett et al, Monoclonal Antibodies and Hybridomas: A New Dimension in Biological Analyses (1980); Campbell, “Monoclonal Antibody Technology” in Laboratory Techniques in Biochemistry and Molecular Biology (1984)). Antibodies produced can be isolated and purified using techniques that are well known in the art (see e.g., Harlow, et al., Antibodies: A Laboratory Manual, Cold Spring Harbor Laboratory, N.Y. (1988)). The antibodies can be used, inter alia, in methods designed to detect the presence of BVD in biological or laboratory samples.

In another embodiment, the present invention is directed to a method of modifying a genome of an isolated wild type BVD virus in such a manner as to make it suitable for use in an immunogenic composition or a vaccine.

According to this method of the present invention, the genomic nucleic acid is modified to mutate the N^{pro} gene, and to insert a sequence coding for a monomeric bovine ubiquitin operably between the mutated N^{pro} coding sequence and the coding sequence of the core protein. The mutation introduced in the N^{pro} gene is one that renders the protein product inactive, i.e., unable to effectively carry out its normal biological function, e.g., proteolytic cleavage between the N and C protein, such that the virus is attenuated by phenotype analysis such as plaque assay and virus growth kinetics on cell culture, yet such mutation does not interfere with the function of the 5′ region of the N^{pro} gene such that the virus can replicate at a desired rate. Attenuated viruses so generated are suitable for use in an immunogenic composition or a vaccine.

In accordance with the present invention, a preferred mutation to be introduced in the N^{pro} coding sequence is a deletion of about 468 bp, more preferably about 194 bp, from the 3′ end of the N^{pro} coding sequence. A particularly preferred mutation is a deletion of about one third of the N^{pro} coding region from the 3′ end (194 bp).

These modifications to the genome of a wild type BVD virus can be made by following procedures well known in the art. For example, genomic RNA can be isolated from a wild type BVD virus, reverse transcribed to form cDNA and then cloned using standard procedures. Mutations can then be introduced into the N^{pro} protease gene by procedures such as the polymerase chain reaction (PCR), site directed mutagenesis, by synthesizing and ligating DNA fragments, or by random mutagenesis techniques including, e.g., exposure to a chemical mutagen or radiation as known in the art, or by a combination of such procedures. Insertion of the ubiquitin coding sequence can be made standard cloning procedures and PCR, for example. The BVD viral genome carrying desired modifications can be cloned into an appropriate vector and produced in large amounts. Either the mutated BVD genome or the vector comprising the genome can be transformed or transfected into a host cell for the purpose of making either large amounts of viral nucleic acid or virus itself.

In a related embodiment of the present invention, methods for making attenuated BVD viruses are provided. In accordance with the present invention, attenuated BVD viruses can be produced which are less infectious than the parent wild type BVD virus, yet replicate at a rate suitable for use as a vaccine or immunogenic composition against BVD infection. In general, the procedure involves isolating a wild type BVD virus; cloning its genomic nucleic acid; modifying the cloned nucleic acid so as to mutate the 3′ region of the N^{pro} protease gene and operably inserting a ubiquitin gene; and then introducing the modified nucleic acid into a host to produce the attenuated virus. The attenuated BVD viruses made by such method, host cells infected with such viruses and progeny attenuated virus produced by these host cells, as well as antibodies made using the attenuated viruses so produced are also encompassed by the present invention.

The attenuated BVD viruses of the present invention, as well as the genomic nucleic acid molecules of such viruses can be used for treating BVDV-caused infections. Accordingly, the present invention further provides compositions and methods useful for treating BVDV-caused infections.

One embodiment of the present invention provides immunogenic compositions which include one or more of the attenuated BVD viruses of the present invention described above. A preferred attenuated BVD virus to be included in an immunogenic composition of the present invention is BVDdN6.

By “immunogenic” is meant the capacity of an attenuated BVD virus in provoking an immune response in an animal against BVD viruses (including both type I and type II BVD viruses), either a cellular immune response mediated primarily by cytotoxic T-cells, or a humoral immune response mediated primarily by helper T-cells which in turn activate B-cells leading to antibody production.

In an alternative embodiment, the immunogenic compositions of the present invention include a genomic nucleic acid molecule of at least one of the attenuated viruses of the present invention.

The immunogenic compositions of the present invention can also include additional active ingredient such as other immunogenic compositions against BVDV, e.g., those described in copending application Ser. No. 08/107,908, WO 9512682, WO 9955366, U.S. Pat. No. 6,060,457, U.S. Pat. No. 6,015,795, U.S. Pat. No. 6,001,613, and U.S. Pat. No. 5,593,873, all of which are incorporated by reference in their entirety.

In addition, the immunogenic compositions of the present invention can include one or more veterinarily-acceptable carriers. As used herein, “a veterinarily-acceptable carrier” includes any and all solvents, dispersion media, coatings, adjuvants, stabilizing agents, diluents, preservatives, antibacterial and antifungal agents, isotonic agents, adsorption delaying agents, and the like. Diluents can include water, saline, dextrose, ethanol, glycerol, and the like. Isotonic agents can include sodium chloride, dextrose, mannitol, sorbitol, and lactose, among others. Stabilizers include albumin, among others. Adjuvants include, but are not limited to, the RIBI adjuvant system (Ribi inc.), alum, aluminum hydroxide gel, oil-in water emulsions, water-in-oil emulsions such as, e.g., Freund's complete and incomplete adjuvants, Block co polymer (CytRx, Atlanta Ga.), SAF-M (Chiron, Emeryville Calif.), AMPHIGEN® adjuvant, saponin, Quil A, QS-21 (Cambridge Biotech Inc., Cambridge Mass.), or other saponin fractions, monophosphoryl lipid A, Avridine lipid-amine adjuvant, heat-labile enterotoxin from E. coli (recombinant or otherwise), cholera toxin, or muramyl dipeptide, among many others. The immunogenic compositions can further include one or more other immunomodulatory agents such as, e.g., interleukins, interferons, or other cytokines.

The immunogenic compositions of the present invention can be made in various forms depending upon the route of administration. For example, the immunogenic compositions can be made in the form of sterile aqueous solutions or dispersions suitable for injectable use, or made in lyophilized forms using freeze-drying techniques. Lyophilized immunogenic compositions are typically maintained at about 4° C., and can be reconstituted in a stabilizing solution, e.g., saline or and HEPES, with or without adjuvant.

The immunogenic compositions of the present invention can be administered to animal subjects to induce an immune response against BVDV. Accordingly, another embodiment of the present invention provides methods of stimulating an immune response against BVDV in an animal subject by administering an effective amount of an immunogenic composition of the present invention described above. By “animal subjects” is meant to include any animal that is susceptible to BVDV infections, such as sheep and swine.

In accordance with the methods of the present invention, a preferred immunogenic composition for administration to an animal subject includes the attenuated virus BVDdN6. An immunogenic composition containing an attenuated BVD virus is administered to a cattle preferably via parenteral routes, although other routes of administration can be used as well, such as e.g., by oral, intranasal, intramuscular, intra-lymph node, intradermal, intraperitoneal, subcutaneous, rectal or vaginal administration, or by a combination of routes.

Immunization protocols can be optimized using procedures well known in the art. A single dose can be administered to animals, or, alternatively, two or more inoculations can take place with intervals of two to ten weeks. The extent and nature of the immune response induced in the cattle can be assessed by using a variety of techniques. For example, sera can be collected from the inoculated animals and tested for the presence of antibodies to BVD virus. Detection of responding CTLs in lymphoid tissues can be achieved by T cell activation assay as indicative of induction of cellular immune response. The relevant techniques are well described in the art, e.g., Coligan et al. Current Protocols in Immunology, John Wiley & Sons Inc. (1994).

Another aspect of the present invention is directed to vaccine compositions.

The term “vaccine” as used herein refers to a composition which prevents or reduces the risk of infection or which ameliorates the symptoms of infection. The protective effects of a vaccine composition against a pathogen are normally achieved by inducing in the subject an immune response, either cell-mediated or humoral immune response or a combination of both. Generally speaking, abolished or reduced incidences of BVDV infection, amelioration of the symptoms, or accelerated elimination of the viruses from the infected subjects are indicative of the protective effects of a vaccine composition.

In one embodiment, the vaccine compositions of the present invention include one or more of the above-described attenuated BVD viruses, preferably BDVdN6. Typically, a vaccine contains between about 1×10⁶ to about 1×10⁸ virus particles, with a veterinarily acceptable carrier, in a volume of between 0.5 and 5 ml. Veterinarily acceptable carriers suitable for use in vaccine compositions can be any of those described hereinabove.

In another embodiment, the vaccine compositions of the present invention include one or more genomic nucleic acid molecules of the attenuated BVD viruses of the present invention. Either DNA or RNA encoding the attenuated BVD viral genome can be used in vaccines. The DNA or RNA molecule can be present in a “naked” form or it can be administered together with an agent facilitating cellular uptake (e.g., liposomes or cationic lipids). The typical route of administration will be intramuscular injection of between about 0.1 and about 5 ml of vaccine. Total polynucleotide in the vaccine should generally be between about 0.1 μg/ml and about 5.0 mg/ml. Polynucleotides can be present as part of a suspension, solution or emulsion, but aqueous carriers are generally preferred. Vaccines and vaccination procedures that utilize nucleic acids (DNA or mRNA) have been well described in the art, e.g., U.S. Pat. No. 5,703,055, U.S. Pat. No. 5,580,859, U.S. Pat. No. 5,589,466, International Patent Publication WO 98/35562, and by Ramsay et al., 1997, Immunol. Cell Biol. 75:360-363; Davis, 1997, Cur. Opinion Biotech. 8:635-640; Manickan et al., 1997, Critical Rev. Immunol. 17:139-154; Robinson, 1997, Vaccine 15(8):785-787; Robinson et al., 1996, AIDS Res. Hum. Retr. 12(5):455-457; Lai and Bennett, 1998, Critical Rev. Immunol. 18:449-484; and Vogel and Sarver, 1995, Clin. Microbiol. Rev. 8(3):406-410, all of which are incorporated herein by reference.

The vaccine compositions of the present invention can also include additional active ingredient such as other vaccine compositions against BVDV, e.g., those described in copending application Ser. No. 08/107,908, WO 9512682, WO 9955366, U.S. Pat. No. 6,060,457, U.S. Pat. No. 6,015,795, U.S. Pat. No. 6,001,613, and U.S. Pat. No. 5,593,873, all of which are incorporated by reference in their entirety.

Vaccination can be accomplished by a single inoculation or through multiple inoculations. If desired, sera can be collected from the inoculated animals and tested for the presence of antibodies to BVD virus.

In another embodiment of the present invention, the above vaccine compositions of the present invention are used in treating BVDV infections. Accordingly, the present invention provides methods of treating BVDV infections in animal subjects by administering to an animal, a therapeutically effective amount of an attenuated BVD virus of the present invention.

By “animal subjects” is meant to include any animal that is susceptible to BVDV infections, such as sheep and swine. By “treating” is meant preventing or reducing the risk of infection by a virulent strain of BVDV (including both Type I and Type II), ameliorating the symptoms of a BVDV infection, or accelerating the recovery from a BVDV infection.

The amount of a virus that is therapeutically effective may vary depending on the particular virus used, the condition of the cattle and/or the degree of infection, and can be determined by a veterinary physician. A preferred virus for use in treating a BVDV infection is BVDdN6.

In practicing the present methods, a vaccine composition of the present invention is administered to a cattle preferably via parenteral routes, although other routes of administration can be used as well, such as e.g., by oral, intranasal, intramuscular, intra-lymph node, intradermal, intraperitoneal, subcutaneous, rectal or vaginal administration, or by a combination of routes. Boosting regiments may be required and the dosage regimen can be adjusted to provide optimal immunization.

The attenuated BVD viruses included in the vaccine compositions of the present invention are distinguished from wild type BVD strains in both the genomic composition and the proteins expressed. Such distinction allows discrimination between vaccinated and infected animals, and permits the identification of the BVDV in the event of alleged vaccine-associated outbreaks. For example, a determination can be made as to whether an animal tested positive for BVDV in certain laboratory tests carries a pathogenic BVD virus, or simply carries an attenuated BVD virus of the present invention previously inoculated through vaccination.

Accordingly, a further aspect of the present invention provides methods of determining the attenuated virus of a prior vaccination as the origin of the BVD virus present in an animal subject.

A variety of assays can be employed for making the determination. For example, the viruses can be isolated from the animal subject tested positive for BVDV, and nucleic acid-based assays can be used to determine the presence of mutations in the N^{pro} gene of the viral genome, or the presence of the ubiquitin coding sequence, which is indicative of an attenuated BVD virus used in a prior vaccination. The nucleic acid-based assays include Southern or Northern blot analysis, PCR, and sequencing. Alternatively, protein-based assays can be employed. For example, cells or tissues suspected of an infection can be isolated from the animal tested positive for BVDV. Intracellular extracts can be made from such cells or tissues and can be subjected to, e.g., Western Blot, using antibodies specific for the deleted portion of N^{pro}. The detection of a signal in such assays can eliminate the possibility that the BVD virus in the animal is from a prior vaccination. Any variations of the foregoing assays are also encompassed by the present invention.

The present invention is further illustrated by the following examples.

### EXAMPLE 1

Construction of Plasmid pBVDdN6

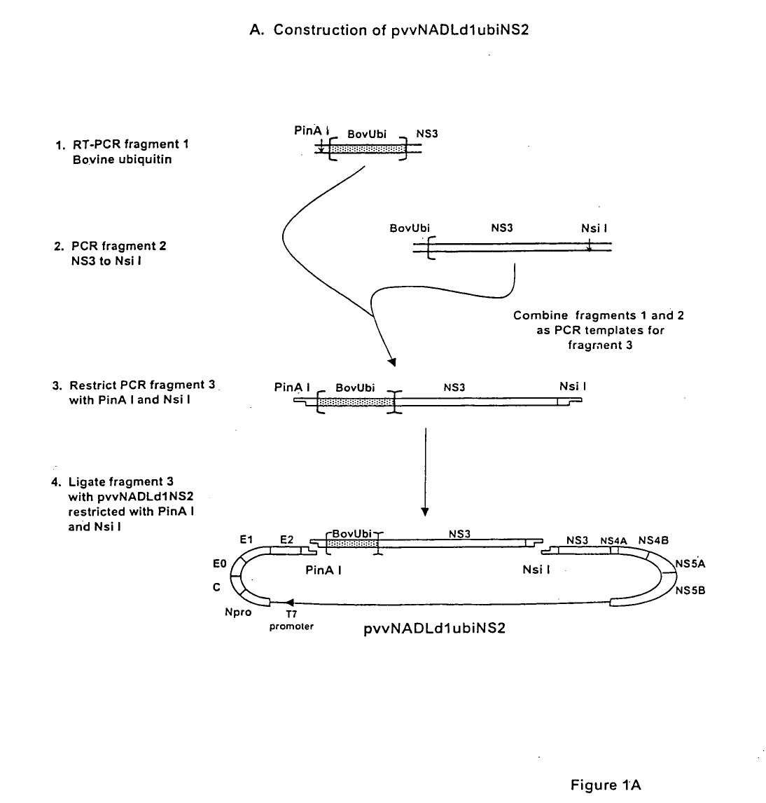

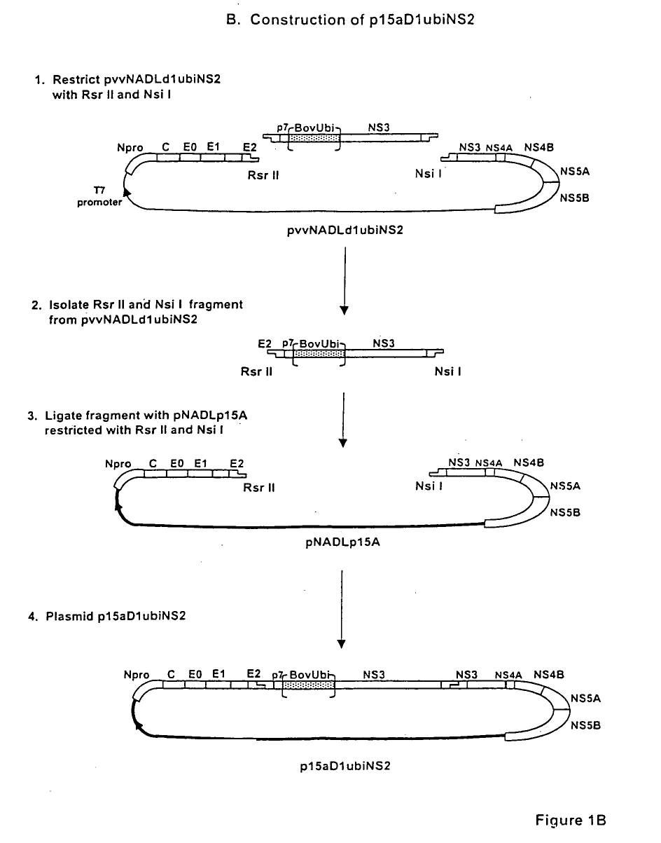

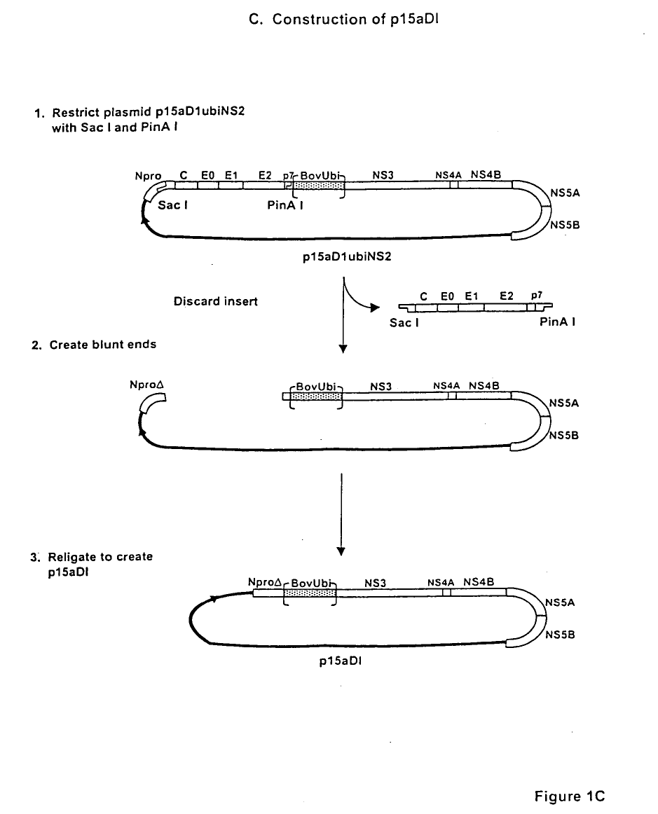

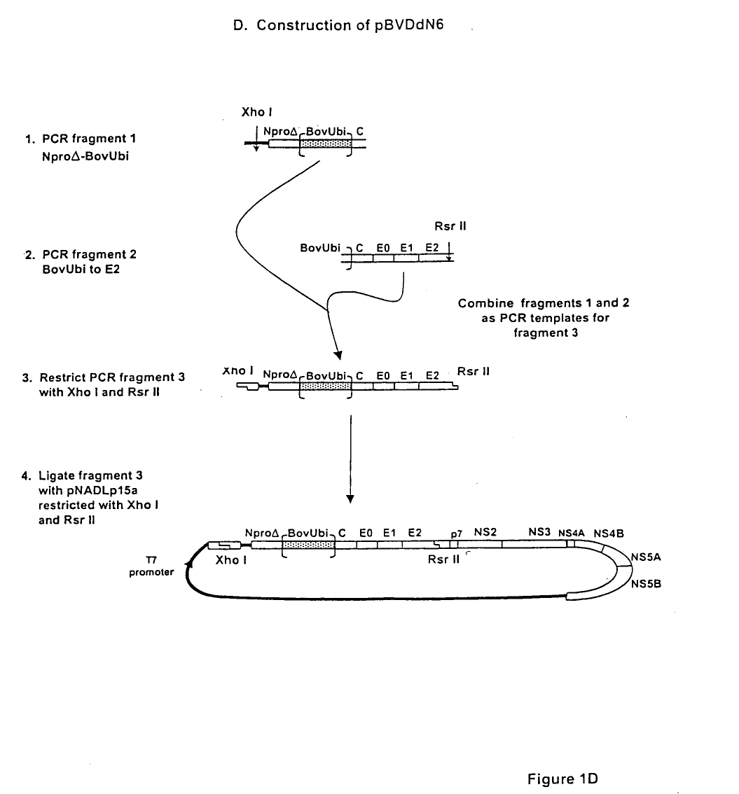

Generation of plasmid pBVDdN6 involved several steps graphically depicted in FIG. 1. Briefly, the coding sequence of bovine ubiquitin gene was cloned into plasmid pvvNADLd1 NS2 giving rise to plasmid pvvNADLd1 ubiNS2 (FIG. 1A). From pvvNADLd1ubiNS2, a fragment containing the coding sequence for bovine ubiquitin and partial BVDV genomic sequences was moved into plasmid pNADLp15a (alternative infectious clone of BVDV) to obtain plasmid p15aD1 ubiNS2 (FIG. 1B). Further modification of plasmid p15aD1ubiNS2 results in plasmid p15aDI (subviral replicon, FIG. 1C) which was subsequently used as the parent plasmid for the generation of plasmid pBVDdN6 (FIG. 1D).

A. Cloning of Bovine Ubiquitin and Construction of pvvNADLd1 ubiNS2.

The DNA sequence of bovine polyubiquitin has been described by Meyers, G., et al. (Virology:180, 602-616, 1991) and is present in GenBank (BOVPOUBA, Accession # M62429 M37794). Cloning and introduction of a monomeric ubiquitin into vector pvvNADLd1NS2 involved two rounds of PCR amplification and synthesis of three PCR fragments. Plasmid pvvNADLd1 NS2 is a derivative of pvvNADL (an infectious clone of BVDV described in U.S. patent application Ser. No. 08/107,908) in which the coding region of NS2 is deleted. In the first round, PCR fragments 1 and 2 were generated which then served as templates for the second round of PCR amplification resulting in PCR fragment 3 (FIG. 1A).

1. Generation of PCR Fragment 1 Encoding a Monomeric Bovine Ubiquitin.

To obtain a template for PCR fragment 1, total cellular RNA was isolated from MDBK cells (a derivative of Madin Darby Kidney bovine kidney cells clone 6). One T-75 tissue culture flask of MDBK cells was lysed using the Ultraspec RNA Isolation System (Biotecx Laboratories, Houston, Tex.) according to the manufacturer's protocol and total cellular RNA was extracted. Oligonucleotide primers for the PCR amplification of fragment 1 were designed to amplify an ubiquitin monomer based on the GenBank sequence for bovine polyubiquitin. The sequences for the two primers were as follows. The 5′ forward primer was GZ51 (+): 5′-CGGACCGGTATGCAGATCTTCGTGAAGACCCTGAC-3′ (SEQ ID NO:1) and the 3′ reverse primer was GZ52(−): 5′-CACGGCAGGCCCACC ACCCCTCAGACGGAGGACCAG-3′ (SEQ ID NO:2). Primer GZ51 (+) annealed to the bovine polyubiquitin sequence at nucleotides 35-60 (GenBank BOVPOUBA sequence) and contained 3 extra nucleotides at the 5′ end which provide stability to the PCR product (GC-clamp) followed by the unique restriction enzyme site PinA 1 (6 nucleotides). Primer GZ52(−) annealed to the bovine polyubiquitin sequence at nucleotides 239 to 262 and had, at the 5′ end, 12 extra nucleotides homologous to the 5′ end of the coding region for BVDVNADL NS3 nucleotides 5423 to 5434.

An aliquot of total cellular RNA (1 ul/50 ul) was reverse-transcribed and PCR amplified with primers GZ51 (+) and GZ52(−) (final concentration 0.5 uM) using Ready-To-Go RT-PCR Beads (Amersham Pharmacia Biotech, Inc. Piscataway, N.J.) according to the protocol supplied by the manufacturer. Fragment 1 generated was 249 base pairs in length (FIG. 1A, step 1).

2. Generation of PCR Fragment 2.

PCR fragment 2 was designed to be homologous to the 5′ half of the coding region for BVDVNADL NS3 and to contain a sequence overlapping with the 3′ end of the ubiquitin sequence in fragment 1 (FIG. 1A, step 2). The sequences of the oligonucleotide primers for fragment 2 were as follows. The 5′ forward primer was GZ53(+): 5′-CTGAGGGGTGGTGGGCCTGCCGTGTGTAAGAAG-3′ (SEQ ID NO:3) and the 3′ reverse primer was GZ54(−): 5′-CCAAGATCCTCCCCTTTCATTACCTCG-3′ (SEQ ID NO:4). Primer GZ53(+) annealed to the coding sequence of BVDVNADL NS3 nucleotides 5423-5443, and had 12 extra nucleotides at the 5′ end which were homologous to the 3′ end of the ubiquitin monomer (nucleotides 251-262). Primer GZ54(−) annealed within the NS3 coding region (nucleotides 6538-6564). PCR amplifications were performed with primers GZ53 and GZ54 at a final concentration of 0.5 uM each, 10 ng of plasmid pNADLp15a as template, 5 units of Pfu DNA polymerase (Stratagene, La Jolla, Calif.). The amplification conditions were: 10 cycles of denaturing at 94° C. for 20 seconds, annealing at 56° C. for 30 seconds, and extension at 72° C. for 2 minutes 30 seconds; then 15 cycles of denaturing at 94° C. for 10 seconds, annealing at 60° C. for 30 seconds, and extension at 72° C. for 2 minutes 30 seconds with autoextension of 20 seconds per cycle. Fragment 2 generated was 1153 base pairs in length.

3. Second Round PCR Amplification and Generation of PCR Fragment 3.

PCR fragments 1 and 2 from round one were purified with a QIAquick PCR purification kit (Qiagen Inc., Valencia, Calif.) and eluted in 50 ul water. PCR amplification for the second round was performed with primers GZ51 and GZ54 at a final concentration of 0.5 uM each, equal volumes of purified fragments 1 and 2 (1 ul each or 3 ul each) as template, and 5 units of Pfu DNA polymerase (Stratagene, La Jolla, Calif.). The amplification conditions were: 10 cycles of denaturing at 94° C. for 20 seconds, annealing at 56° C. for 30 seconds, and extension at 72° C. for 2 minutes 30 seconds; followed by 15 cycles of denaturing at 94° C. for 10 seconds, annealing at 60° C. for 30 seconds, and extension at 72° C. for 2 minutes 30 seconds with an autoextension of 20 seconds per cycle (FIG. 1A, step 3).

After PCR, the resulting fragment of 1378 base pairs was purified using the QIAquick PCR purification Kit (Qiagen Inc. Valencia, Calif.), eluted in 50 ul water and digested with restriction enzymes PinA I and Nsi I (Roche Molecular Biochemicals, Indianapolis, Ind.). The digested fragment, 1117 base pairs in size, was agarose gel purified and eluted with GENECLEAN (BIO101, Vista, Calif.) glassmilk.

4. Preparation of Vector Plasmid and Ligation.

Vector plasmid pvvNADLd1 NS2 contained a deletion of nucleotides 3821 to 4993 in the NS2 coding region and had a unique PinA I restriction site (5′-ACCGGT-3′, SEQ ID No:5, coding for amino acids threonine and glycine) inserted at the site of the deletion. Plasmid pvvNADLd1 NS2 clone#7 DNA (13,411 base pairs in size) was digested with Nsi I and PinA I (Roche Molecular Biochemicals, Indianapolis, Ind.), treated with calf intestinal alkaline phosphatase (New England Biolabs, Inc., Beverly, Mass.) and agarose gel purified. The digested 12,096 base pair long vector fragment was eluted using GENECLEAN (BIO101, Vista, Calif.) glassmilk.

The PinA I and Nsi I digested PCR fragment 3 was mixed with cleaned vector fragment at an approximate molecular ratio of 10:1 and ligated with 2,000 U T4 DNA ligase (New England Biolabs, Inc., Beverly, Mass.) at 16° C. overnight. STBL2 E. coli cells (Gibco/BRL) were transformed with an aliquot of the ligation reaction and heterologous colonies which represented different populations of DNA plasmids were screened by mini-DNA purification and specific restriction enzyme digestion. Plasmids of expected size (13,214 bp) were further confirmed by sequence analysis. The resulting plasmid pvvNADLd1 ubiNS2 contained a deletion of NS2 sequences and an insertion of monomeric bovine ubiquitin directly upstream of the coding region for NS3 (at nucleotide 5423) (FIG. 1A, Step 4).

B. Construction of Plasmid p15aD1ubiNS2.

As observed previously, amplification of clone pwNADL in E. coli was difficult since the plasmid was unstable during propagation in E. coli (Vassilev et al., J. Virol. 71: 471-478, 1997). To continue with the further construction of plasmid pBVDdN6 it was necessary to obtain a more stable plasmid. Therefore a part of plasmid pvvNADLd1 ubiNS2 which included bovine ubiquitin and flanking sequences encompassing the NS2 deletion was moved into the more stable plasmid pNADLp15a to obtain plasmid p15aD1ubiNS2 (FIG. 1B).

1. Description of parent plasmid pNADLp15A.

Infectious full-length clone NADLp15A clone 4 was generated by subcloning the entire BVDV genome of molecular clone pvvNADL (described in U.S. application Ser. No. 08/107,908) into intermediate-copy number p15a vector pACYC177 (New England Biolabs, Inc. GenBank Accession #: X06402). Briefly, pACYC177 was digested with restriction enzyme Hae II to obtain a 2510 base pair fragment which was ligated with a 14,209 base pair fragment derived from Hae II-digested plasmid pvvNADL. This resulted in clone pNADLp15a which was 16,719 base pairs in length and had improved stability. pNADLp15a and all derivatives of this construct were propagated in E. coli strain GM2163 (New England Biolabs, Inc., Beverly, Mass.). Transcription of BVDV RNA from this plasmid was directed by a T7 RNA polymerase promoter inserted immediately upstream of the BVDV genome. The sequence of the BVDV genome in the full-length clones pvvNADL and pNADLp15A was derived from the National Animal Disease Laboratory (NADL) strain of BVDV (American Type Culture Collection VR-534).

Plasmids pNADLp15a and pvvNADLd1ubiNS2 clone #7 were digested with unique restriction enzymes Rsr II and Nsi I (New England Biolabs, Inc., Beverly, Mass.) (FIG. 1B, step 1). The 13,240 bp fragment of pNADLp15a and the 2,541 bp fragment of pvvNADLd1 ubiNS2 clone #7 were purified and ligated (as described for pvvNADLd1 ubiNS2) to obtain plasmid p15aD1ubiNS2 (FIG. 1B, steps 2 to 4). RNA transcribed from this plasmid and transfected into MDBK cells supported viral RNA replication in immunohistochemical assays which detected viral protein E2, but did not give rise to infectious virus particles.

C. Construction of Plasmid p15aDI with a N^{pro}-Ubiquitin Fusion.

To prepare the fusion of the ubiquitin sequence with the partial N^{pro} sequence, plasmid p15aD1ubiNS2 was further modified. The sequences for all structural genes including part of the 3′ coding region of the amino-terminal protease N^{pro} were deleted (FIG. 1C). Plasmid p15aD1ubiNS2 was digested with restriction enzyme Sac I (nucleotide 699) which cleaved within the N^{pro} coding region and with PinA I which cut at the 5′ end of the ubiquitin coding region. To create blunt ends, the reaction was treated with Pfu I DNA polymerase for 30 minutes at 70° C. (FIG. 1C, steps 1 and 2). The resulting 12,228 base pair fragment was agarose gel purified and ligated (step 3). An aliquot of the ligation reaction was used to transform E coli strain GM2163 by electroporation. Transformants were subject to a PCR using a primer with a T7 promoter, which amplified the sequence encompassing the N^{pro}-ubiquitin fusion region. The resulting PCR fragment was in vitro translated in a TNT T7 Quick rabbit reticulocyte system (Promega, Madison Wis.) in the presence of ³⁵S-methionine (Trans-label from Amersham). Clones with the correct deletion and in-frame fusion of N^{pro}-ubi were expected to give rise to a translation product of approximately 22 kD in size. Clones were considered positive if the expected product was detected after SDS-PAGE and autoradiography. This construct was termed as p15aDI.

RNA transcribed from p15aDI and transfected into MDBK cells also supported viral RNA replication in immunohistochemical assays which detected viral protein NS3, but did not give rise to infectious virus particles.

D. Generation of Construct pBVDdN6.

Plasmid p15aDI contained a partial N^{pro} coding sequence fused to ubiquitin and lacked all structural genes of BVDV including the coding region for NS2. To generate the intended construct pBVDdN6 which could produce infectious virus particles, the structural genes including the coding region for NS2 were reintroduced downstream of the N^{pro}-ubiquitin fusion sequence. For construction of vector pBVDdN6, PCR fragments 1 and 2 were generated in the first round of PCR amplification. These fragments then served as templates in the second round of PCR amplification to generate PCR fragment 3 (FIG. 1D).

1. Generation of PCR Fragment 1

PCR fragment 1 was designed to amplify a region of p15aDI which spanned from the 5′NTR of the BVDV coding region to the end of the ubiquitin coding region (FIG. 1D, step 1). The sequences for the two primers were as follows. The 5′ forward primer was GZ68(+): 5′-GGAATAAAGGTCTCGAGATGCCAC-3′ (SEQ ID NO: 6) and the 3′ reverse primer was GZ66(−): 5′-CTTTCGTGTCTGAACCACCCCTCAGACGGAGGACC-3′ (SEQ ID NO: 7). Primer GZ68(+) annealed to the 5′NTR of the BVDV sequence at nucleotides 218 to 237 and included a unique Xho I site which was also present in the BVDV genome. Primer GZ66(−) annealed to the 3′ end of the ubiquitin sequence (nucleotides 241-262, GenBank BOVPOUBA sequence numbering) and had 13 extra nucleotides at the 5′ end which were homologous to the 5′ end of the coding region for BVDVNADL Core protein (C) (nucleotides 890 to 902). PCR amplifications were performed with primers GZ68(+) and GZ66(−) at final concentrations of 0.3 uM each, 10 ng of plasmid p15aDI as template, and with 5 units of Pfu DNA polymerase (Stratagene, La Jolla, Calif.). Amplification conditions were: 10 cycles of denaturing at 94° C. for 15 seconds, annealing at 60° C. for 30 seconds, and extension at 72° C. for 45 seconds; followed by 15 cycles of denaturing at 94° C. for 15 seconds, annealing at 62° C. for 30 seconds, and extension at 72° C. for 45 seconds with autoextensions of 5 seconds per cycle. The resulting Fragment 1 was 716 base pairs in length.

2. Generation of PCR Fragment 2

PCR fragment 2 was designed to be homologous to the 5′ end of the coding region for BVDVNADL Core coding region and to contain a sequence overlapping with the ubiquitin sequence in fragment 1 (FIG. 1D, step 2). The sequences of the oligonucleotide primers for amplifying fragment 2 were as follows. The 5′ forward primer was GZ67(+): 5′-CCGTCTGAGGGGTGGTTCAGACACGAAAGAAGAGGGAG-3′ (SEQ ID NO: 8) and the 3′ reverse primer was SEQ24(−): 5′-GCCTTGCCTATGAGGGAATGG-3′ (SEQ ID NO: 9). Primer GZ67(+) annealed to BVDVNADL C coding region (nucleotides 890 to 911) and had 16 extra nucleotides at the 5′ end which were homologous to the 3′ end of the ubiquitin monomer (nucleotides 250-262). Primer SEQ24(−) annealed within the E2 coding region (nucleotides 2942-2962). PCR amplifications were performed with primer pairs at a final concentration of 0.3 uM each, 10 ng of plasmid pNADLp15a as template, and with 5 units of Pfu DNA polymerase (Stratagene, La Jolla, Calif.). Amplification conditions were: 10 cycles of denaturing at 94° C. for 15 seconds, annealing at 58° C. for 30 seconds, and extension at 68° C. for 3 minutes; followed by 15 cycles of denaturing at 94° C. for 15 seconds, annealing at 62° C. for 30 seconds, and extension at 68° C. for 3 minutes with autoextension of 20 seconds per cycle. This fragment was 2089 base pairs in length.

3. Second round of PCR amplification and generation of PCR fragment 3.

PCR fragments 1 and 2 from the first amplification were purified with QIAquick PCR purification kit (Qiagen Inc., Valencia, Calif.) and eluted with 50 ul water. The second round of PCR amplification was performed with primers GZ68(+) and SEQ24(−) (see above) at a final concentration of 0.3 uM with 5 units of Pfu DNA polymerase (Stratagene, La Jolla, Calif.). Equal volumes of purified fragments 1 and 2 were combined (1 ul each) to serve as PCR template (FIG. 1D, Step 3). The amplification conditions were: 10 cycles of denaturing at 94° C. for 15 seconds, annealing at 60° C. for 30 seconds, and extension at 72° C. for 5 minutes 36 seconds; followed by 15 cycles of denaturing at 94° C. for 15 seconds, annealing at 62° C. for 30 seconds, and extension at 72° C. for 5 minutes 36 seconds with an autoextension of 20 seconds per cycle. PCR fragment 3 was 2784 base pairs in length.

4. Restriction Enzyme Digestion, Ligation and Screening.

The QIAquick kit purified fragment 3 was digested with the unique restriction enzymes Xho I and Rsr 11 (2644 bp fragment). Vector pNADLp15A was also digested with Xho I and Rsr 11 (14,119 bp). The PCR fragment and the vector were both agarose gel purified and eluted using GENECLEAN (BIO101, Vista, Calif.) glassmilk.

Digested PCR fragment and vector fragment were mixed at an estimated molecular ratio of 5:1 to 10:1 and ligated using 2,000 U T4 DNA ligase (New England Biolabs, Inc., Beverly, Mass.) at 16° C. overnight. GM2163 E. coli cells (Gibco/BRL) were transformed with an aliquot of the ligation reaction and heterologous colonies which represented different populations of DNA plasmids were screened by mini-DNA purification and specific restriction enzyme digestion. Plasmids of the expected size of 16,763 bp were further confirmed by sequence analysis. The resulting plasmid pBVDdN6 had the partial coding sequence of N^{pro} with a deletion in the 3′ region, fused to bovine ubiquitin which was directly upstream of the coding region for Core protein starting at nucleotide 890 (FIG. 1D, step 4). The full-sequence of pBVDdN6 is shown in FIG. 2B.

### EXAMPLE 2

### Characterization of the BVDdN6 Viral Clone

In Vitro transcription and RNA transfection:

RNA transcripts were synthesized in vitro with T7 RNA polymerase using MEGAscript™ reagent (Ambion) according to the manufacture's protocol. All BVDV-carrying DNA templates were linearized with Ksp I and treated with T4 DNA polymerase to remove the 3′ overhang. The products of the transcription reaction were analyzed by gel electrophoresis. One to five μg of transcript RNA was added to 200 μl of Opti-MEM (GibcoBRL) containing 6 μg of Lipofectin (Gibco-BRL). RNA/Lipids samples were incubated for 10 to 15 min at room temperature. During this time, monolayers (50 to 60% confluent) of MDBK (a derivative of Madin Darby Kidney cells clone 6) cells grown in six-well plates (35 mm diameter) were washed twice with RNase-free PBS, once with Opti-MEM. After the final wash, the transfection RNA/Lipids mixtures were added to each cell well and the wells were then incubated for 10 min at room temperature with gentle rocking. Opti-MEM of 1 ml was then added to each of the cell wells with transfection mixtures, and the wells were incubated for another three hours at 37° C. A 3-ml volume of Opti-MEM containing 2-3% bovine donor calf serum (CDS) was added to each of the wells. Following incubation for two to four days at 37° C., the cells were either fixed with 80% acetone and subject to an immunohistochemistry assay for visualizing the BVDV plaques, or collected for further analysis using either MDBK or RD cells. RD is a stable transformed bovine testis cell line which was normally culture in Opti-MEM medium with 5% fetal equine serum (FES).

Infectivity of pBVDdN6

RNA from pBVDdN6 and pNADLp15A (positive control) was synthesized in vitro as described above. RNA transfection was performed using Lipofectin on MDBK cell monolayers. At 24 and 48 hrs post-transfection, one set of total transfected cell monolayers were collected to reinfect fresh MDBK monolayers for generating virus stocks, another duplicate set of the transfected cell monolayers were fixed with 80% acetone for immunohistochemistry assay. Immunohistochemistry was done with Vectastain Elite ABC kit (Vector laboratories) according to the manufacturer's instructions. A Monoclonal antibody (CA3) against the BVD-specific viral protein E2 was used in 1:1000 dilution. Viruses (termed as BVDdN6 virus) were recovered after transfection of RNA derived from pBVDdN6 DNA nearly as soon as after transfection of RNA derived from pNADLp15A. Envelop protein E2 was detected and virus was produced at 24 hrs post-transfection with RNAs derived from both pNADLp15A and pBVDdN6 DNAs.

Phenotype Analysis

In order to characterize the nature of the rescued virus BVDdN6, early passage virus stocks (passage 2) were inoculated onto MDBK cell monolayers. For comparison, the wild type NADL virus (passage 2) and the BVDdN1 virus (passage 2) were inoculated onto MDBK cell monolayers as well. At different post-infection times as 16, 24, 32 and 48 hrs, the cell monolayers were fixed with 80% acetone. The infected cells were detected in an immunohistochemistry assay using monoclonal antibody CA3 against viral protein E2 at 1:1000 dilution and was examined with microscope.

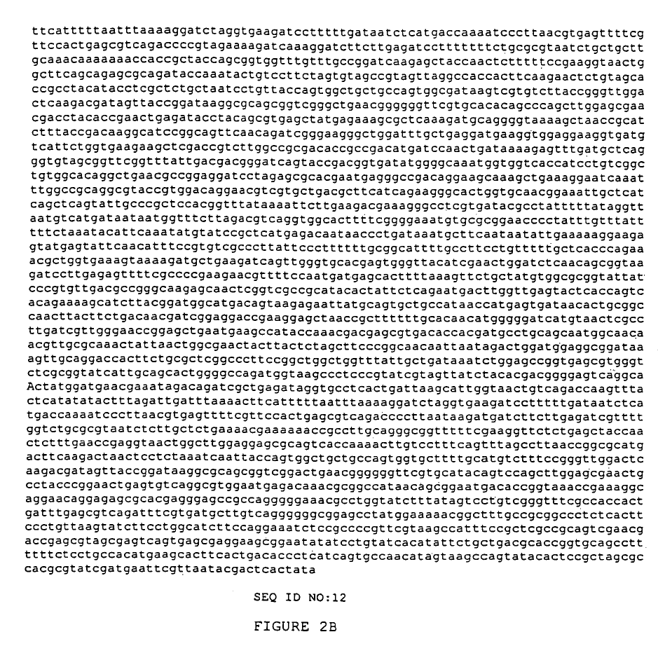

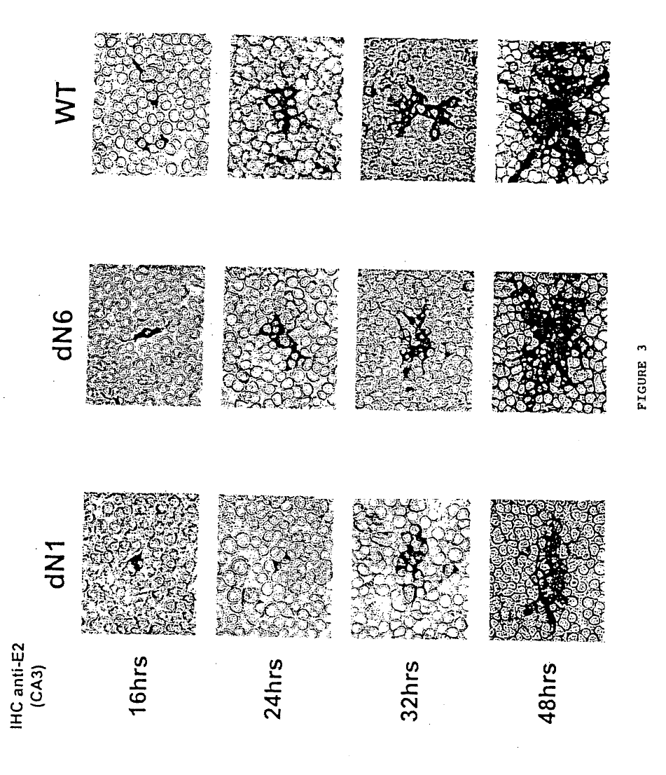

As shown in FIG. 3, although all three viruses were detectable at 16 hrs post-infection, both the BVDdN6 virus and the wild type NADL virus replicated faster than the BVDdN1 virus, and the second round infection to the neighbor cells were observed in both wild type and BVDdN6 infected cells. At 48 hrs post-infection, the size of the infected cell cluster from BVDdN1 was much smaller than that of either BVDdN6 or wild type. The cluster of cells infected with the wild type virus was slightly large than that with the BVDdN6 infection.

This result indicated that the virus BVDdN6 replicated much faster than the attenuated virus BVDdN1.

Genotype Analysis

The genome of the BVDdN6 virus was analyzed to confirm the partial deletion of the N^{pro} gene and the insertion of the bovine ubiquitin gene. Viral RNAs of all three viruses, BVDdN1, BVDdN6 and wild type (passage 3) were purified from infected RD monolayers using Ultraspec™ RNA reagent (Biotect) following the manufacturer's instruction. RT/PCR experiments were performed using oligonucleotides NADLC4(−) and GZ68(+) and the STEP™ RT-PCR system (GibcoBRL). NADLC4(−) had the sequence 5′-GCTATTATTGCCCACGCCAACAATGC-3′ (SEQ ID NO: 10) (Negative sense, oligonucleotides 1142-1167). This primer annealed to a region at around 30 bp from the C-terminal of the core protein C. GZ68(+) had the sequence 5′-GGAATAAAGGTCTCG AGATGCCAC-3′ (SEQ ID NO: 6) (positive sense, oligonucleotides 213-236). GZ68(+) annealed to a region near the 5′ end of the viral genome. RT/PCR from parental RNA (wt), BVDdN6 RNA and BVDdN1 RNA yielded a fragment of 950 bp, 989 bp and 446 bp, respectively, as expected.

The RT/PCR fragment from BVDdN6 viral RNA was also subject to sequence analysis. Viral RNA of BVNdN6 virus had the sequence as constructed (see FIGS. 1D and 2).

Growth Kinetics of BVDdN6, BVDdN1 and wt NADL

The growth kinetics of the viruses BVDdN6, BVDdN1 and wt NADL in MDBK cells were compared. Subconfluent monolayers in 12 well plates were infected at a multiplicity of infection of 1.0. Viruses were adsorbed for one hour. Before cells were supplied with fresh medium, the first sample at time point zero was collected. Virus titers were determined from the total mixture of supernatants and cell lysates. To determine the virus titer in cells, cells with the supernatants were freeze/thawed three times at −80 C. Virus titers (log TCID₅₀ per millilitre) were determined at 0, 4, 8, 12, 16, 20, 24, 36, 48, 60 and 72 hrs. after infection. Virus titration was performed on RD cells in 96 wells and the positive, infected cells were determined in an immunohistochemistry assay using mAb CA3 specific for the envelope protein E2.

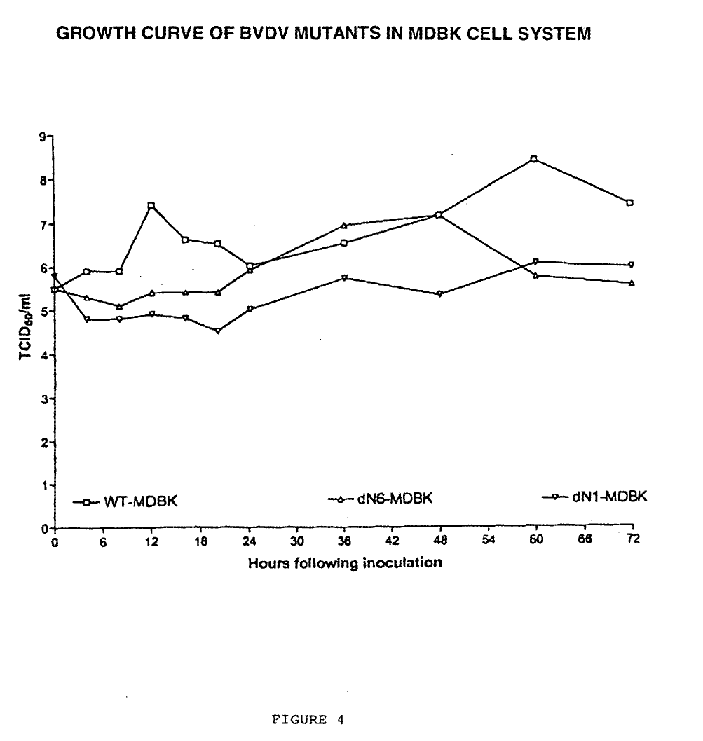

As shown in FIG. 4, the BVDdN6 virus grew slower than the wild type virus, and faster than the BVDdN1 virus. This result was consistent with the observation from the phenotype analysis, shown in FIG. 3.

TABLE 1
Sequence of SEQUENCE ID NOS. 1-10 (5′—3′)

| | |
|---|---|
| SEQUENCE ID NO 1: | |
| Seq1 GZ51(+) | cggaccggtatgcagatcttcgtgaagaccctgac |
| SEQUENCE ID NO 2: | |
| Seq2 GZ52(−) | cacggcaggcccaccacccctcagacggaggaccag |
| SEQUENCE ID NO 3: | |
| Seq3 GZ53(+) | ctgaggggtggtgggcctgccgtgtgtaagaag |
| SEQUENCE ID NO 4: | |
| Seq4 GZ54(−) | ccaagatcctcccctttcattacctcg |
| SEQUENCE ID NO 5: | |
| Seq5 | accggt |
| SEQUENCE ID NO 6: | |
| Seq6 GZ68(+) | ggaataaaggtctcgagatgccac |
| SEQUENCE ID NO 7: | |
| Seq7 GZ66(−) | ctttcgtgtctgaaccacccctcagacggaggacc |
| SEQUENCE ID NO 8: | |
| Seq8 GZ67(+) | ccgtctgaggggtggttcagacacgaaagaagagggag |
| SEQUENCE ID NO 9: | |
| Seq9 SEQ24(−) | gccttgcctatgagggaatgg |
| SEQUENCE ID NO 10: | |
| Seq10 NADLC4(−) | gctattattgcccacgccaacaatgc |
| SEQUENCE ID NO 11: | |
| Seq11 | the genomic sequence of BVDdN6 (nucleotides 1-12617 |
| | are shown in FIG. 2A) |
| SEQUENCE ID NO 12: | |
| Seq12 | pBVDdN6 (FIG. 2B) |

1. An attenuated bovine viral diarrhea (BVD) virus, wherein said virus carries in the viral genome, a mutated N^{pro} coding sequence comprising an intact 5′ region, and a sequence coding for a monomeric bovine ubiquitin, wherein the ubiquitin coding sequence is operably placed between the 3′ end of said mutated N^{pro} coding sequence and the 5′ end of the coding sequence for the viral core protein.

2. The attenuated BVD virus of claim 1, comprising a genomic nucleic acid sequence as set forth in SEQ ID NO: 11, or a degenerate variant thereof.

3. The attenuated BVD virus of claim 1, comprising a genomic nucleic acid sequence essentially as set forth in SEQ ID NO: 11, or a degenerate variant thereof.

4. An isolated nucleic acid molecule comprising the genomic sequence of an attenuated BVD virus, wherein said virus carries in the viral genome, a mutated N^{pro} coding sequence comprising an intact 5′ region, and a sequence coding for a monomeric bovine ubiquitin, wherein the ubiquitin coding sequence is operably placed between the 3′ end of said mutated N^{pro} coding sequence and the 5′ end of the coding sequence for the viral core protein.

5. An isolated nucleic acid molecule comprising a sequence as set forth in SEQ ID NO: 11, or a degenerate variant thereof.

6. An isolated nucleic acid molecule comprising a sequence essentially as set forth in SEQ ID NO: 11, or a degenerate variant thereof.

7. A vector comprising a sequence essentially as set forth in SEQ ID NO: 11, or a degenerate variant thereof.

8. A vector designated as pBVDdN6 (ATCC No. PTA-2532) (SEQ ID NO: 12).

9. A cell transformed or transfected with any of the nucleic acid molecules of claims 4-6.

10. A cell transformed or transfected with the vector of claim 7 or 8.

11. A progeny virus produced by the cell of claim 9.

12. A progeny virus produced by the cell of claim 10.

13. A method of modifying the genomic nucleic acid molecule of an isolated wild type BVD virus, comprising introducing a mutation into the 3′ region of the N^{pro} protease gene wherein said mutation renders the N^{pro} protein inactive, and inserting a sequence coding for a monomeric bovine ubiquitin between the mutated N^{pro} coding sequence and the coding sequence of the core protein.

14. The method of claim 13, wherein said mutation in the N^{pro} protease gene comprises a deletion of about one third of the N^{pro} coding region from the 3′ end.

15. A method of attenuating an isolated wild type BVD virus, comprising isolating the genomic nucleic acid molecule from said virus, introducing a mutation into the 3′ region of the N^{pro} protease gene in the viral genome, wherein said mutation renders the N^{pro} protein inactive; inserting a sequence coding for a monomeric bovine ubiquitin between the mutated N^{pro} coding sequence and the coding sequence of the core protein; and producing from the modified genome an attenuated virus suitable for use in a vaccine.

16. An immunogenic composition comprising the attenuated BVD virus of any of claims 1-3 and a veterinarily-acceptable carrier.

17. An immunogenic composition comprising the isolated nucleic acid molecule of any of claims 4-6 and a veterinarily-acceptable carrier.

18. A method of inducing an immune response against BVDV in an animal subject, comprising administering an immunologically effective amount of the attenuated BVD virus of any of claims 1-3 and a veterinarily-acceptable carrier.

19. A method of inducing an immune response against BVDV in an animal subject, comprising administering an immunologically effective amount of the isolated nucleic acid molecule of any of claims 4-6.

20. The method of claim 18, wherein said immune response is a cellular or humoral immune response.

21. The method of claim 19, wherein said immune response is a cellular or humoral immune response.

22. The method of claim 18, wherein said immune response results in the production of antibodies against BVDV in said animal.

23. The method of claim 19, wherein said immune response results in the production of antibodies against BVDV in said animal.

24. A vaccine composition comprising the attenuated BVD virus of any of claims 1-3 and a veterinarily-acceptable carrier.

25. A vaccine composition comprising the isolated nucleic acid molecule of any of claims 4-6 and a veterinarily-acceptable carrier.

26. A method of treating a BVDV infection in an animal, comprising administering to said animal, a therapeutically effective amount of the attenuated BVD virus of any of claims 1-3.

27. A method of treating a BVDV infection in an animal, comprising administering to said animal, a therapeutically effective amount of the isolated nucleic acid molecule of any of claims 4-6.

28. A method of identifying a BVD virus in an animal as an attenuated BVD virus of any of claims 1-3, said animal suspected of suffering a BVDV infection, comprising isolating the virus from said animal, detecting the presence of the ubiquitin coding sequence, thereby determining the isolated virus as identical to the attenuated BVD virus of any of claims 1-3.

29. A method of identifying a BVD virus in an animal as an attenuated BVD virus of any of claims 1-3, said animal suspected of suffering a BVDV infection, comprising isolating the virus from said animal, detecting the presence of the mutation in the N^{pro} gene, thereby determining the isolated virus as identical to the attenuated BVD virus of any of claims 1-3.
